(19) United States
(12) Patent Application Publication
(10) Pub. No.: US 2008/0090892 A1
(43) Pub. Date: Apr. 17, 2008

(54) AMORPHOUS ASENAPINE AND PROCESSES FOR PREPARING SAME

(75) Inventors: Melissa Jean Casteel, Norwich, CT (US); Wilhelmus Petrus Hubertus Maria de Wildt, Oss (NL)

Correspondence Address: ORGANON USA, INC.;PATENT DEPARTMENT, 56 LIVINGSTON AVENUE, ROSELAND, NJ, 07068, US

(73) Assignee: N.V. Organon, Oss (NL)

(21) Appl. No.: 11/868,361

(22) Filed: Oct. 5, 2007

Related U.S. Application Data
(60) Provisional application No. 60/828,535, filed on Oct. 6, 2006.

(51) Int. Cl. A61K 31/407; A61P 25/18; C07D 487/04

(52) U.S. Cl. 514/410; 548/421

(57) ABSTRACT

This disclosure relates to amorphous asenapine and pharmaceutically acceptable complexes, salts, solvates and hydrates thereof, to solid pharmaceutical compositions containing it, and to its use to treat central nervous system disorders, including schizophrenia and bipolar disorder. This disclosure also relates to methods and materials for preparing amorphous asenapine and pharmaceutical compositions which contain it.

### BACKGROUND OF THE INVENTION

This invention relates to amorphous asenapine, to solid pharmaceutical compositions containing it, and to its use to treat central nervous system (CNS) disorders, including schizophrenia and bipolar disorder. This invention also relates to methods and materials for preparing amorphous asenapine and pharmaceutical compositions which contain it.

Asenapine, or trans-5-chloro-2-methyl-2,3,3a,12b-tetrahydro-1H-dibenz[2,3:6,7]-oxepino[4,5-c]pyrrole, is described in U.S. Pat. No. 4,145,434 to van den Burg and is represented by a structure of Formula 1: 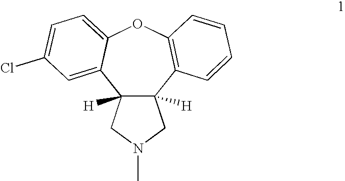
The pharmacological profile of asenapine, its kinetics and metabolism, and the first safety and efficacy studies in human volunteers are reviewed in De Boer et al., Drugs of the Future, 18(12):1117-1123 (1993). Asenapine is a broad-spectrum, high potency serotonin, noradrenaline and dopamine antagonist. As described in published patent application WO 99/32108, asenapine exhibits potential antipsychotic activity and may be useful for treating depression.

The maleate salt of asenapine is currently undergoing clinical evaluation. As described in Funke et al., Arzneim.-Forsch./Drug Res., 40:536-539 (1999), the earliest known form of asenapine maleate (Form H) is a monoclinic crystalline form having a melting point in the range of 141° C. to 145° C. Patent application PCT/EP2006/061480 describes the discovery of a new form of asenapine maleate (Form L), which is an orthorhombic crystalline form having a melting point in the range of 138° C. to 142° C.

Asenapine is typically dosed through the oral mucosa—i.e., via sublingual and buccal administration. See, e.g., published patent application WO 95/23600. For this reason, physical forms of asenapine having an increased in vitro dissolution rate would be desirable.

### SUMMARY OF THE INVENTION

This invention provides amorphous asenapine and pharmaceutically acceptable complexes, salts, solvates, and hydrates thereof, which may be used in pharmaceutical compositions, including those suitable for sublingual and buccal administration. Amorphous asenapine may be used to treat a variety of CNS disorders or conditions, including schizophrenia and other psychotic disorders, mood disorders, and combinations of these disorders or conditions.

One aspect of the invention provides a compound selected from trans-5-chloro-2-methyl-2,3,3a,12b-tetrahydro-1H-dibenz[2,3:6,7]oxepino[4,5-c]pyrrole and pharmaceutically acceptable complexes, salts, solvates, and hydrates thereof. The compound is at least 50%, 75%, 90%, 95%, or 99% amorphous, based on the total weight of the compound.

Another aspect of the invention provides a compound which is a maleic acid salt of trans-5-chloro-2-methyl-2,3,3a,12b-tetrahydro-1H-dibenz[2,3:6,7]oxepino[4,5-c]pyrrole and is at least 50%, 75%, 90%, 95%, or 99% amorphous, based on the total weight of the compound.

The compound may be characterized by one or more of the following: (a) a ¹³C solid state nuclear magnetic resonance spectrum having chemical shifts in parts per million (ppm) of 169.9, 136.4, 129.5, and 42.6, the chemical shifts referenced to an external standard of solid adamantane at 29.5 ppm; (b) an X-ray powder diffraction pattern obtained with CuKα radiation having a single broad peak between 2θ values of about 15° and about 30°; and (c) a glass transition onset temperature of about 38° C. to about 53° C.

An additional aspect of the invention provides a method of making a compound selected from trans-5-chloro-2-methyl-2,3,3a,12b-tetrahydro-1H-dibenz[2,3:6,7]oxepino[4,5-c]-pyrrole and pharmaceutically acceptable complexes, salts, solvates, and hydrates thereof. The compound is at least 50%, 75%, 90%, 95%, or 99% amorphous, based on the total weight of the compound. The method comprises: (a) forming a liquid solution comprising a solvent and the compound; (b) atomizing the liquid solution into droplets; and (c) removing at least a portion of the solvent to form the compound. Here, step (a) may include dissolving a precursor of the compound in the solvent, the precursor having the same chemical structure as the compound but less amorphous content, e.g., crystalline trans-5-chloro-2-methyl-2,3,3a,12b-tetrahydro-1H-dibenz[2,3:6,7]oxepino[4,5-c]pyrrole maleate.

### BRIEF DESCRIPTION OF THE DRAWINGS

FIG. 1 shows an XRPD (1.54 Å) diffractogram for a sample of crystalline asenapine maleate (Form L), which was used to prepare amorphous asenapine maleate.

FIG. 2 shows an XRPD (1.54 Å) diffractogram for a sample of amorphous asenapine maleate (Batch A1), which was prepared by spray drying.

FIG. 3 shows a thermogram for a sample of crystalline asenapine maleate (Form L), which was used to prepare amorphous asenapine maleate.

FIG. 4 shows thermograms for a sample of amorphous asenapine maleate (Batch A2), during heating segments 3, 6, and 9 of a 9-step temperature program.

FIG. 5 shows a ¹³C ssNMR spectrum collected using CPMAS experiments for a sample of crystalline asenapine maleate (Form L), which was used to prepare amorphous asenapine maleate.

FIG. 6 shows a ¹³C ssNMR spectrum collected using CPMAS experiments for a sample of amorphous asenapine maleate (Batch A1), which was prepared by spray drying.

### DETAILED DESCRIPTION

### Definitions and Abbreviations

Unless otherwise indicated, this disclosure uses definitions provided below.

“About,” “approximately,” and the like, when used in connection with a numerical variable, generally refers to the value of the variable and to all values of the variable that are within the experimental error (e.g., within the 95% confidence interval for the mean) or within ±10% of the indicated value, whichever is greater.

“Subject” refers to a mammal, including a human.

“Pharmaceutically acceptable” refers to substances, which are within the scope of sound medical judgment, suitable for use in contact with the tissues of subjects without undue toxicity, irritation, allergic response, and the like, commensurate with a reasonable benefit/risk ratio, and effective for their intended use.

“Treating” refers to reversing, alleviating, inhibiting or slowing the progress of, or preventing a disorder or condition to which such term applies, or to preventing one or more symptoms of such disorder or condition.

“Treatment” refers to the act of “treating.”

“Drug,” “drug substance,” “active pharmaceutical ingredient,” and the like, refer to a compound that may be used for treating a subject in need of treatment.

“Excipient” or “adjuvant” refers to any component of a pharmaceutical composition that is not the drug substance.

“Drug product,” “pharmaceutical dosage form,” “final dosage form,” and the like, refer to the combination of one or more drug substances and one or more excipients (i.e., pharmaceutical composition) that is administered to a subject in need of treatment, and may be in the form of tablets, capsules, liquid suspensions, patches, and the like.

“Inert” refers to substances that may positively influence the bioavailability of the drug, but are otherwise unreactive.

“Amorphous” refers to any solid substance which (i) lacks order in three dimensions, or (ii) exhibits order in less than three dimensions, order only over short distances (e.g., less than 10 Å), or both. Thus, amorphous substances include partially crystalline materials and crystalline mesophases with, e.g. one- or two-dimensional translational order (liquid crystals), orientational disorder (orientationally disordered crystals), or conformational disorder (conformationally disordered crystals). Amorphous solids may be characterized by known techniques, including X-ray powder diffraction (XRPD) crystallography, solid state nuclear magnet resonance (ssNMR) spectroscopy, differential scanning calorimetry (DSC), or some combination of these techniques. As illustrated, below, amorphous solids give diffuse XRPD patterns, typically comprised of one or two broad peaks (i.e., peaks having base widths of about 5° 2θ or greater).

“Crystalline” refers to any solid substance exhibiting three-dimensional order, which in contrast to an amorphous solid substance, gives a distinctive XRPD pattern with sharply defined peaks.

“Solid dispersion,” “amorphous solid dispersion,” and the like, refer to a drug substance, which has been dispersed or distributed in a carrier or dispersion medium. Generally, at least a portion, and in many cases a majority, of the drug substance is amorphous. The drug may be present in the dispersion as (a) discrete, drug-rich domains or may be (b) homogeneously distributed throughout the carrier (i.e., a solid solution) or may be some combination of (a) and (b). For a discussion of pharmaceutical solid dispersions, see W. L. Chiou & S. Riegelman, J. Pharm. Sci 60(9):1282-1302 (1971), which is herein incorporated by reference.

“Particle size” refers to the median or to the average dimension of particles in a sample and may be based on the number of particles, the volume of particles, or the mass of particles, and may be obtained using any number of standard measurement techniques, including laser diffraction methods, centrifugal sedimentation techniques, photon correlation spectroscopy (dynamic light scattering or quasi-elastic light scattering), or sieving analysis using standard screens. Unless stated differently, all references to particle size in this specification refer to the mean particle size based on volume.

“Solvate” describes a molecular complex comprising the drug substance and a stoichiometric or non-stoichiometric amount of one or more pharmaceutically acceptable solvent molecules (e.g., ethanol). When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex will often be non-stoichiometric.

“Hydrate” describes a solvate comprising the drug substance and a stoichiometric or non-stoichiometric amount of water.

TABLE 1 lists abbreviations used throughout the specification.

TABLE 1
List of Abbreviations

| Abbreviation | Description |
|---|---|
| Å | Angstrom unit |
| CAP | cellulose acetate phthalate |
| CMC | Carboxymethylcellulose |
| CMEC | Carboxymethylethylcellulose |
| CNS | central nervous system |
| CPMAS | cross-polarization magic angle spinning |
| d10, d50, d90 | cumulative distribution functions in which 10%, |
| | 50% and 90% of the solids (based on volume) have |
| | diameters less than d10, d50, and d90, respectively |
| DSC | differential scanning calorimetry |
| EC | ethyl cellulose |
| HEC | hydroxyethyl cellulose |
| HPC | Hydroxypropylcellulose |
| HPMC | Hydroxypropylmethylcellulose |
| HPMCAS | hydroxypropylmethylcellulose acetate succinate |
| HPMCAT | hydroxypropylmethylcellulose acetate trimellitate |
| HPMCP | hydroxypropylmethylcellulose phthalate |
| ICH | International Committee on Harmonization |
| MC | Methylcellulose |
| PLM | polarized light microscopy |
| PVP | Polyvinylpyrrolidone |
| RH | relative humidity |
| SCFM | standard cubic feet per minute |
| ssNMR | solid state nuclear magnetic resonance |
| Tg | glass transition temperature |
| w/w | weight (mass)/total weight (mass) × 100, % |
| XRPD | X-ray powder diffraction |

Amorphous asenapine may be prepared from crystalline asenapine (or suitable precursor) by spray drying, spray coating, lyophilization, and other methods. Spray drying and spray coating both involve dissolving asenapine in a compatible solvent, atomizing the resulting solution, and evaporating the solvent to form drug substance comprised of amorphous asenapine. Lyophilization or freeze drying also involves dissolving asenapine in a compatible solvent (usually water), and includes rapidly freezing the solution to form amorphous asenapine and removing the solvent via sublimation (typically under vacuum) and desorption. For more detailed description of lyophilization, see Georg-Wilhelm Oetjen, “Freeze-Drying,” Ullmann's Encyclopedia of Industrial Chemistry (2004).

For each of these methods, the fraction of drug substance that is amorphous is in the range of about 50% w/w to about 100% w/w, 75% w/w to about 100% w/w, 90% w/w to about 100% w/w, or about 95% w/w to about 100% w/w, based on the total mass of asenapine. Ideally, the fraction of asenapine that is amorphous is in the range of about 99% w/w to about 100% w/w, based on the total mass of asenapine.

Asenapine may be prepared in various ways. For example, U.S. Pat. No. 4,145,434 to van den Burg describes a general methodology for preparing asenapine. Vader et al., J. Labeled Comp. Radiopharm., 34:845-869 (1994) describes additional synthetic methods for preparing asenapine and its radiolabeled derivatives. More recently, patent application PCT/EP2006/061409 and U.S. Provisional Patent Application 60/806,583 describe improved methods for preparing asenapine and its pharmaceutically acceptable complexes, salts, solvates, and hydrates. Patent application PCT/EP2006/061480 describes the preparation of the orthorhombic crystalline form of asenapine maleate (Form L).

Amorphous asenapine may be prepared using any pharmaceutically acceptable form of asenapine, including its free base and its pharmaceutically acceptable complexes, salts, solvates, and hydrates. Useful salts may include acid addition salts (including di-acids) including nontoxic salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride, hydrobromide, hydroiodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts. For a discussion of other useful salts, see S. M. Berge et al., “Pharmaceutical Salts,” 66 J. Pharm. Sci., 1-19 (1977); see also Stahl and Wermuth, Handbook of Pharmaceutical Salts: Properties, Selection, and Use (2002).

Pharmaceutically acceptable salts of asenapine may be prepared using various methods. For example, one may react the free base of asenapine with an appropriate acid to give the desired salt. One may also react a precursor of the compound of asenapine with an acid or base to remove an acid- or base-labile protecting group. Additionally, one may convert a salt of the compound of Formula 1 to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

Asenapine may also exist in unsolvated and solvated forms, including hydrates, and in the form of multi-component complexes (other than salts and solvates) in which asenapine and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun., 17:1889-1896 (2004). For a general review of multi-component complexes, see J. K. Haleblian, J. Pharm. Sci. 64(8):1269-88 (1975).

Amorphous asenapine may be prepared using any form of asenapine, including its crystalline polymorphs, e.g., Form L, Form H, and mixtures of Form L and Form H. Thus, one advantage of using amorphous asenapine is that it may be prepared without having to purify the starting crystalline material.

Amorphous asenapine may be prepared from the trans-stereoisomer shown in Formula 1, above, which may be stereoisomerically pure or may be a mixture of stereoisomers. With reference to Formula 1, above, asenapine may exist as a single enantiomer having absolute (S,S)-stereochemical configuration as indicated by the wedged bonds. Alternatively, asenapine may exist as a mixture of the (S,S)- and the (R,R)-enantiomers (e.g., racemate) having the relative stereochemical configuration indicated by the wedged bonds. Asenapine may also exist as a single enantiomer having the opposite absolute (R,R)-stereochemical configuration, in which the two stereogenic centers shown in Formula 1 are inverted.

In addition, the pharmaceutical composition may employ prodrugs of asenapine. Such prodrugs may be prepared by replacing appropriate functional groups of asenapine with functionalities known as “pro-moieties,” as described, for example, in H. Bundgaar, Design of Prodrugs (1985).

Useful forms of asenapine may also include pharmaceutically acceptable isotopically labeled compounds in which one or more atoms are replaced by atoms having the same atomic number, but an atomic mass or mass number different from the atomic mass or mass number that predominates in nature. Examples of isotopes suitable for inclusion in asenapine include isotopes of hydrogen (²H and ³H), carbon (¹¹C, ¹³C and ¹⁴C), oxygen (¹⁵O, ¹⁷O and ¹⁸O), nitrogen (¹³N and ¹⁵N), and chlorine (³⁶Cl). Isotopically labeled forms of asenapine may be prepared by techniques known to those skilled in the art.

As noted above, amorphous asenapine may be prepared by spray drying, which includes dissolving crystalline asenapine in one or more compatible solvents to form a solution. A compatible solvent is any liquid which will dissolve asenapine. In practice, a compatible solvent includes any liquid which, at room temperature, will completely dissolve asenapine at a concentration of about 1% w/w or greater, or more typically, at a concentration of about 5% w/w or greater. Useful solvents include those which are volatile, have a normal boiling point of about 150° C. or less, exhibit relatively low toxicity, and can be removed from amorphous asenapine so that the level of solvent in the drug product meets the International Committee on Harmonization (ICH) guidelines for residual solvent. Additional processing, such as tray-drying, may be required to meet ICH residual solvent levels.

Useful solvents include water; alcohols such as methanol, ethanol, n-propanol, isopropanol, and various isomers of butanol; ketones, such as acetone, methyl ethyl ketone, methyl iso-butyl ketone, and cyclohexanone; esters, such as methyl acetate, ethyl acetate, and propyl acetate; ethers, such as dimethyl ether, tetrahydrofuran, methyl tetrahydrofuran, 1,3-dioxolane, and 1,4-dioxane; alkanes, such as butane and pentane; alkenes, such as pentene and cyclohexene; nitriles, such as acetonitrile; alkyl halides, such as methylene chloride, trichloroethane, chloroform, and trichloroethylene; aromatics, such as toluene; and mixtures thereof. Lower volatility solvents such as dimethyl acetamide, dimethylformamide, or dimethylsulfoxide can also be used in small amounts in mixtures with a volatile solvent. Mixtures of solvents, such as 50% methanol and 50% acetone, can also be used, as can mixtures with water. Particularly useful solvents include acetone, methyl ethyl ketone, methyl isobutyl ketone, methanol, ethanol, n-propanol, isopropanol, methyl acetate, ethyl acetate, toluene, methylene chloride, tetrahydrofuran, 1,4-dioxane, 1,3-dioxolane, and mixtures thereof. Especially useful solvents include acetone, methanol, ethanol, n-propanol, isopropanol, ethyl acetate, and mixtures thereof. Mixtures of the above with water may also be used.

The solution may be prepared by adding asenapine to the solvent with concurrent or subsequent mixing. Mixing may be carried out using mechanical means, e.g., through the use of overhead mixers, magnetically driven mixers or stirring bars, planetary mixers, or homogenizers. The solvent and drug substance may be combined at room temperature or the solvent may be heated to aid in dissolution of asenapine.

Asenapine may be added to the solvent up to its solubility limit in the solvent. To ensure complete dissolution, however, the amount of asenapine added is usually less than about 80% of its solubility limit at the solution temperature. The concentration of asenapine typically ranges from about 0.1% w/w to about 30% w/w depending on the solubility of the drug and the amount of any additional excipients. The concentration of asenapine in the solution is typically at least about 0.1%, 0.5%, 1%, or 5% w/w. Although increasing the concentration of asenapine reduces the volume of solvent that is evaporated to form amorphous asenapine, higher concentrations of asenapine may be too viscous to atomize efficiently into small droplets. A solution viscosity of about 0.5 cp to about 50,000 cp or about 10 cp to about 2,000 cp generally results in satisfactory atomization.

In addition to solvent and drug substance, the solution may contain optional excipients so long as asenapine remains in solution. The optional excipients may be dissolved in the solution, may be suspended in the solution, or may be dissolved and suspended in the solution. Useful excipients included matrix-forming agents.

Matrix-forming agents may help stabilize amorphous asenapine, preventing or retarding formation of crystalline asenapine, or may improve the properties of amorphous asenapine for processing into final dosage forms. The matrix-forming agent may be polymeric or non-polymeric, and may comprise several components. Thus the matrix-forming agent may comprise two or more polymeric components, two or more non-polymeric components, or a combination of polymeric and non-polymeric components.

The term “polymeric” means a compound that is made of monomers connected together to form a larger molecule. A polymeric component generally comprises at least about 20 monomers. Thus, the molecular weight of a polymeric component will generally be about 2000 daltons or more. The polymeric component may be neutral or ionizable, and may be cellulosic or non-cellulosic. In general, useful polymers have an aqueous-solubility of at least about 0.1 mg/mL. Examples of neutral non-cellulosic polymers include vinyl polymers and copolymers, polyvinyl alcohols, polyvinyl alcohol/polyvinyl acetate copolymers, polyethylene glycol/polypropylene glycol copolymers, polyvinyl pyrrolidone, polyethylene/polyvinyl alcohol copolymers, and polyoxyethylene/polyoxypropylene block copolymers (also known as poloxamers). Examples of ionizable non-cellulosic polymers include carboxylic acid functionalized polymethacrylates and carboxylic acid functionalized polyacrylates, amine-functionalized polyacrylates and polymethacrylates, proteins such as gelatin and albumin, and carboxylic acid functionalized starches such as starch glycolate. Examples of neutral cellulosic polymers are hydroxypropyl methyl cellulose (HPMC), hydroxyethyl cellulose (HEC), hydroxypropyl cellulose (HPC), methyl cellulose, hydroxyethyl methyl cellulose, and hydroxyethyl ethyl cellulose. Examples of ionizable cellulosic polymers are hydroxypropyl methyl cellulose acetate succinate (HPMCAS), hydroxypropyl methyl cellulose phthalate (HPMCP), carboxymethyl ethyl cellulose (CMEC), carboxyethyl cellulose, carboxymethyl cellulose, cellulose acetate phthalate (CAP), hydroxypropyl methyl cellulose acetate phthalate, and cellulose acetate trimellitate.

The term “non-polymeric” means that the component is not polymeric. Exemplary non-polymeric materials for use as a matrix-forming agents include: organic acids and their salts, such as stearic acid, citric acid, fumaric acid, tartaric acid, malic acid, and pharmaceutically acceptable salts thereof, long-chain fatty acid esters, such as glyceryl monooleate, glyceryl monostearate, glyceryl palmitostearate, polyethoxylated castor oil derivatives, hydrogenated vegetable oils, glyceryl dibehenate, and mixtures of mono-, di-, and tri-alkyl glycerides; glycolized fatty acid esters, such as polyethylene glycol stearate and polyethylene glycol distearate; polysorbates; salts such as sodium chloride, potassium chloride, lithium chloride, calcium chloride, magnesium chloride, sodium sulfate, potassium sulfate, sodium carbonate, and magnesium sulfate; amino acids such as alanine and glycine; sugars such as glucose, sucrose, xylitol, fructose, lactose, trehalose, mannitol, sorbitol, and maltitol; alkyl sulfates such as sodium lauryl sulfate and magnesium lauryl sulfate; and phospholipids, such as lecithin; and mixtures thereof.

As noted above, the solution comprising asenapine and solvent is delivered to an atomizer that breaks the solution into small droplets. Useful atomizers include “pressure” or single-fluid nozzles; two-fluid nozzles; centrifugal or spinning-disk atomizers; ultrasonic nozzles; and mechanical vibrating nozzles. Detailed descriptions of atomization processes can be found in Lefebvre, Atomization and Sprays (1989), and in Perry's Chemical Engineers' Handbook (7th ed. 1997). Generally, the droplets produced by the atomizer are less than about 500 μm in diameter when they exit the atomizer.

Once atomized, at least a portion of the solvent is removed from the solution to produce a plurality of solid particles comprising amorphous asenapine. The amount of solvent removed to form solid particles depends on the solubility of asenapine in the solvent and the concentration of asenapine and any optional excipients in the solution prior to atomization. Generally, at least about 60% w/w of the solvent originally present in the solution is removed to form solid particles. The greater the amount of solvent removed from the solution, the less likely that crystalline asenapine is formed. Thus, the amount of solvent removed from the solution to form amorphous asenapine is typically at least 70% w/w, at least 80% w/w, or at least 90% w/w.

In addition, increasing the rate of solvent removal typically increases the fraction of amorphous asenapine in the drug substance. In some cases, removing at least 90% w/w of the solvent in five minutes or less may generate asenapine which is 50% w/w amorphous. Removing 90% w/w of the solvent in 60 seconds or less, in 20 seconds or less, or in 10 seconds or less may provide higher fractions of amorphous asenapine.

Atomization and solvent removal occur in a chamber where process conditions may be controlled. The driving force for solvent removal is generally provided by maintaining the partial pressure of the solvent in the chamber below the vapor pressure of the solvent at the temperature of the drying droplets. This may be accomplished by maintaining a partial vacuum in the chamber (e.g., total pressure of about 0.01 atmospheres to about 0.50 atmospheres), by mixing the liquid droplets with a warm drying gas, or both. Some of the energy required for evaporation of solvent may be provided by heating the solution prior to atomization, though generally the energy comes primarily from the drying gas. The solution temperature may range from just above the solvent's freezing point to about 20° C. or more above its normal boiling point, which is achieved by pressurizing the solution. Solution flow rates through the atomizer may vary depending on the type of nozzle, the size of the chamber, and the drying conditions, which include the inlet temperature and the flow rate of the drying gas through the chamber.

The drying gas may, in principle, be essentially any gas, but for safety reasons and to minimize undesirable oxidation of asenapine, the process typically employs an inert gas such as nitrogen, nitrogen-enriched air or argon. The drying gas is generally introduced into the chamber at a temperature of about 60° C. to about 300° C. or about 80° C. to about 240° C.

The large surface-to-volume ratio of the droplets and the large driving force for evaporation of solvent leads to rapid solidification times for the droplets. Solidification times of about 20 seconds or less, of about 10 seconds or less, or of about 1 second or less are typical. Rapid solidification helps maintain uniformity and homogeneity of amorphous asenapine within and among particles.

The particles of amorphous asenapine may remain in the chamber for about 5 seconds to about 60 seconds following solidification, during which time additional solvent evaporates from the particles. Generally, the solvent level of amorphous asenapine as it exits the chamber is less than about 10% w/w and is often less than 2% w/w. Following formation, amorphous asenapine may be dried to remove residual solvent using a suitable process, including tray drying, fluid bed drying, microwave drying, belt drying, rotary drying, or vacuum drying. After drying, residual solvent level is typically less than about 1% w/w and is often less than about 0.1% w/w.

The resulting spray-dried amorphous asenapine is usually in the form of small particles. The mean (volume) diameter of the particles may be less than about 1000 μm, less than about 500 μm, less than about 100 μm, less than about 50 μm, or less than about 25 μm. The size of amorphous asenapine particles may be determined by sieve analysis, microscopy, light scattering, or sedimentation. Useful equipment for measuring particle size includes Coulter Counters, Malvern Particle Size Analyzers, and the like. See, e.g., Remington: The Science and Practice of Pharmacy (20th ed., 2000).

In addition to mean diameter, the size of the particles may be characterized by its “span”

$$span = \frac{{d}_{90} - {d}_{10}}{{d}_{50}} ,$$

where d₅₀ is the diameter of particles that make up 50% of the total volume of particles of equal or smaller diameter; d₉₀ is the diameter of particles that make up 90% of the total volume of particles of equal or smaller diameter; and d₁₀ is the diameter of particles that make up 10% of the total volume of particles of equal or smaller diameter. Span is sometimes referred to as the Relative Span Factor (RSF) and is a dimensionless parameter that measures the uniformity of the particle size distribution. Generally, the lower the span, the narrower the size distribution, which results in improved flow characteristics of the particles. The span of amorphous asenapine particles may be less than about 3, less than about 2.5, or less than about 2.0.

For a general description of spray-drying processes and spray-drying equipment, see Perry's Chemical Engineers' Handbook, pages 20-54 to 20-57 (6th ed., 1984). Further details of spray drying processes and equipment may be found in Marshall, “Atomization and Spray-Drying,” Chem. Eng. Prog. Monogr. Series 2, 50 (1954); see also, Masters, Spray Drying Handbook (4th ed., 1985) and U.S. Pat. No. 6,763,607.

Amorphous asenapine may also be made by spray coating or layering amorphous asenapine onto a core. Spray coating includes dissolving crystalline asenapine in a solvent as described above, and atomizing the resulting solution into droplets which are sprayed onto a core. The solvent is removed from the droplets on the core, forming one or more solid layers of amorphous asenapine on the core. Spray coated cores of amorphous asenapine have the additional advantage of providing large, dense particles which are less likely to become segregated during manufacture than pure amorphous material. In addition, coated cores also have round surfaces and narrow size distributions, which improve the flow characteristics and handling of the amorphous particles.

The core may be pharmaceutically inert and is mainly intended for carrying the layer or layers of amorphous asenapine. The core may be a solid particle or object, which does not disintegrate in the relevant body fluid. Alternatively, the core may comprise a disintegrating agent that will cause the layered particle to rapidly disintegrate in the relevant body fluid (e.g., saliva in the oral cavity). Examples of core materials are non-pareil seeds, sugar beads, wax beads, glass beads, lactose, microcrystalline cellulose, polymer beads, starch, colloidal silica, etc. The core may be made by known methods, such as melt- or spray-congealing, extrusion-spheronization, granulation, spray-drying and the like. Alternatively, the core may be a dosage form such as a tablet, pill, multiparticulate or capsule, which may contain asenapine or a different drug. Spray coating amorphous asenapine onto the dosage form may be useful for a combination therapy of asenapine and another drug.

The cores may have any shape, size, and size distribution suitable for the production of the desired layered particle. In one embodiment, the core is generally spherical with a smooth surface. In another embodiment, the cores range in size of from about 1 μm to about 3000 μm, or from about 10 μm to about 1000 μm, or from about 50 μm to about 500 μm. To obtain a uniform final product it is generally desirable to use cores with a narrow size distribution. The core may be an agglomerate, a granule, or a particle that has been coated with one or more layers or amorphous asenapine. Agglomerates and granules may be made by any method conventionally used in the art, such as extrusion-spheronization, rotary granulation, melt-congealing, spray-drying, vacuum drying, or spray granulation.

A variety of equipment may be used for spray coating the core with amorphous asenapine, including pan coaters, fluidized bed coaters, and rotary granulators. As with spray drying, atomization, and usually solvent removal, occurs in a chamber where process conditions may be controlled. Spray coating processes generally employ one or nozzles, which are placed at the top, along the sidewalls, or at the bottom of the chamber, to atomize the solution into droplets that settle on the cores. Useful nozzles include pressure nozzles, two-fluid nozzles, and three-fluid nozzles.

During coating and solvent evaporation, the cores may be suspended in a gas. For example, in one embodiment, the core particles are carried upwards from the bottom of the spraying chamber by a stream of gas and contact one or more small droplets ejected from a nozzle located at the top of the chamber. In another embodiment, the spray solution is directed in the same direction as the suspending gas.

After spray-coating the cores, solvent is removed from the cores to obtain a deposit or layer of amorphous asenapine on each of the cores. As noted above, at least a portion of the solvent is removed while the particles are in the chamber. In one embodiment, the suspending gas carries the cores through a spraying zone to an evaporation zone where the solvent is removed. In such cases, the gas serves to suspend and dry the particles. During evaporation, the amount of solvent removed is sufficient to prevent the particles from adhering to one another upon exiting the chamber.

Immediately upon exiting the chamber or after interim storage, the particles may undergo additional spray coating and solvent evaporation until the particles reach a predetermined particle size or weight. The determination of the desired particle size or weight may be conducted using known classification techniques. Alternatively, a predetermined amount of the cores is sprayed with a predetermined amount of solution to produce particles having the desired particle size or weight or having a desired amount of asenapine per mass of cores. Typically, the coated cores have a final size of about 3 mm or less, about 2 mm or less, or about 1 mm or less, and have a span of about 3 or less, about 2.5 or less, or about 2 or less.

Amorphous asenapine may undergo further processing to prepare solid pharmaceutical compositions, including final dosage forms such as tablets, capsules, powders, creams, transdermal patches, depots, and the like. For example, drug particles may be used directly to make drug product, or may be milled to a median particle size of, e.g., about 1 μm to about 150 μm. Useful milling equipment includes jet mills (dry), ball mills, hammer mills, and the like. The milled particles may then be combined with additional pharmaceutically acceptable excipients. The resulting mixture may be dry blended (say, in a v-cone blender) to form a drug product, which may optionally undergo further operations, such as tableting or encapsulation, coating, and the like, to prepare the final dosage form of the drug product. For a discussion of milling, dry blending, tableting, encapsulation, coating, and the like, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997).

For tablet dosage forms, depending on dose, the drug may comprise about 1% w/w to about 80% w/w of the dosage form, but more typically comprises about 5% w/w to about 60% w/w of the dosage form. In addition to asenapine, the tablets may include one or more disintegrants, surfactants, glidants, lubricants, binding agents, diluents, flavorants, and sweeteners, either alone or in combination. Examples of disintegrants include sodium starch glycolate; CMC, including its sodium and calcium salts; croscarmellose, including its sodium salt; crospovidone, including its sodium salt; PVP, MC; microcrystalline cellulose; one- to six-carbon alkyl-substituted HPC; starch; pregelatinized starch; sodium alginate; and mixtures thereof. If present, the disintegrant may comprise about 1% w/w to about 30% w/w of the dosage form, or more typically, about 5% w/w to about 25% w/w of the dosage form.

Tablets may optionally include surfactants, such as sodium lauryl sulfate and polysorbate 80; glidants, such as silicon dioxide and talc; lubricants, such as magnesium stearate, calcium stearate, zinc stearate, sodium stearyl fumarate, sodium lauryl sulfate, and mixtures thereof, flavorants, such as menthol and levomenthol; and sweeteners, such as saccharin, sodium saccharin, and acesulfame potassium. When present, surfactants may comprise about 0.2% w/w to about 5% w/w of the tablet; glidants may comprise about 0.2% w/w to about 1% w/w of the tablet; lubricants may comprise about 0.25% w/w to about 10% w/w, or more typically, about 0.5% w/w to about 3% w/w; flavorants may comprise about 0.25% w/w to about 3% w/w; and sweeteners may comprise about 1% w/w to about 50% w/w of the tablet.

As noted above, tablet formulations may include binders and diluents. Binders are generally used to impart cohesive qualities to the tablet formulation and typically comprise about 10% w/w or more of the tablet. Examples of binders include, without limitation, microcrystalline cellulose, gelatin, sugars, polyethylene glycol, natural and synthetic gums, PVP, pregelatinized starch, HPC, and HPMC. One or more diluents may make up the balance of the tablet formulation. Examples of diluents include, without limitation, lactose monohydrate, spray-dried lactose monohydrate, anhydrous lactose, and the like; mannitol; xylitol; dextrose; sucrose; sorbitol; microcrystalline cellulose; starch; dibasic calcium phosphate dihydrate; and mixtures thereof.

Amorphous asenapine may be used to treat CNS disorders, including schizophrenia and other psychotic disorders, mood disorders, and combinations thereof. The standards for diagnosis of these disorders may be found in the American Psychiatric Association's Diagnostic and Statistical Manual of Mental Disorders (4th ed., 2000), which is commonly referred to as the DSM Manual.

For the purposes of this disclosure, schizophrenia and other psychotic disorders include schizophreniform disorder, schizoaffective disorder, delusional disorder, brief psychotic disorder, shared psychotic disorder, psychotic disorder due to general medical condition, and substance-induced psychotic disorder, as well as medication-induced movement disorders, such as neuroleptic-induced Parkinsonism, neuroleptic malignant syndrome, neuroleptic-induced acute dystonia, neuroleptic-induced acute akathisia, neuroleptic-induced tardive dyskinesia, and medication-induced postural tremor.

Mood disorders include depressive disorders, such as major depressive disorder, dysthymic disorder, premenstrual dysphoric disorder, minor depressive disorder, recurrent brief depressive disorder, postpsychotic depressive disorder of schizophrenia, and major depressive episode with schizophrenia; bipolar disorders, such as bipolar I disorder, bipolar II disorder, cyclothymia, and bipolar disorder with schizophrenia; mood disorders due to general medical condition; and substance-induced mood disorders.

Substance-induced disorders refer to those resulting from the using, abusing, dependence on, or withdrawal from, one or more drugs or toxins, including alcohol, amphetamines or similarly acting sympathomimetics, caffeine, cannabis, cocaine, hallucinogens, inhalants, nicotine, opioids, phencyclidine or similarly acting arylcyclohexylamines, and sedatives, hypnotics, or anxiolytics, among others.

For administration to human patients, the total daily dose of the claimed and disclosed compounds is typically in the range of about 0.1 mg to about 3000 mg depending on the route of administration. For example, sublingual and buccal administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient whose mass falls outside of this weight range.

### EXAMPLES

The following examples are intended to be illustrative and non-limiting, and represent specific embodiments of the present invention.

Preparation of Amorphous Asenapine Maleate Via Spray Drying

Amorphous asenapine maleate was prepared by spray drying a solution of asenapine maleate with a PSD Mini Spray Dryer. The solution was prepared by adding acetone (150 mL) to an Erlenmeyer flask containing asenapine maleate (Form L, 2.25 g). The flask was placed on a stir plate, stirred until all solid material was dissolved, then sonicated for 10 minutes. The solution was delivered to the spray dryer via a syringe pump at a feed rate of 1.3 mL/minute. The spray dryer nitrogen flow was set to 1.0 SCFM and the inlet temperature was set to 70° C. Solids were collected on filter paper (WHATMAN, type 1) in two batches. The first batch (A1) was isolated after approximately 100 mL of solution had been spray dried. Solids were isolated from the filter paper and transferred to a crystallization dish. During isolation, the texture of the batch changed from a chalky powder to a tacky semi-solid. A sample was evaluated by polarized light microscopy and the remainder of the batch was placed in the refrigerator overnight. The second batch (A2) exhibited the same change in texture as it was isolated from the filter paper and transferred to a glass bottle. Batch A2 was stored overnight in a capped bottle under ambient conditions. Both batches were subsequently dried in a room temperature vacuum oven (approximately 15 mm Hg) with nitrogen purge for approximately 48 hours. After drying, the material appeared to be a dry powder. Batch A1 and A2 were transferred to glass bottles and sealed in pouches containing desiccant and oxygen scavengers. This transfer was carried out in the vacuum oven under nitrogen purge. The sealed pouches were placed in the freezer for storage.

Characterization of Amorphous Asenapine Maleate

Polarized Light Microscopy (PLM): Photomicrographs of samples of the starting material (Form L) and Batch A1 were generated using an OLYMPUS BX60 optical microscope with a 10× optical path magnification and a halogen light source. Immersion oil was used to disperse a small amount of sample on a glass microscope slide. The microscope was configured with one or more of the following objectives: UPlanFI 4×/0.13, LMPlanFI 10×/0.25, LMPlanFI 20×/0.40, and LMPlanFI 50×/0.50. Samples were viewed under cross-polarized light with a 530 nm wave plate. Data were collected and analyzed using IMAGE-PRO PLUS software Version 4.1.0.0.

PLM analysis indicates that the starting material comprises small (approximately 50 μm diameter) birefringent particles, which appear to contain no amorphous material. PLM analysis indicates that Batch A1 comprises comparatively larger, non-birefringent particles that appear to be completely amorphous.

X-ray Powder Diffraction (XRPD): Diffractograms of samples of the starting material (Form L) and Batch A1 were generated using a SIEMENS D5000 diffractometer using CuKα radiation (1.54 Å). The instrument was equipped with a line focus X-ray tube. The tube voltage and amperage were set to 38 kV and 38 mA, respectively. The divergence and scattering slits were set at 1 mm, and the receiving slit was set at 0.6 mm. Diffracted radiation was detected by a SOL-X energy dispersive X-ray detector. For crystalline asenapine, a theta two theta continuous scan at 2.4° 2θ/minute (1 second/0.04° 2θ step) from 3.0° to 40° 2θ was used. For amorphous asenapine, a theta two theta continuous scan at 2.4° 2θ/minute (0.5 seconds/0.08° 2θ step) from 3.0° to 40° 2θ was used. An alumina standard (NIST standard reference material 1976) was analyzed to check the instrument alignment. Data were collected and analyzed using BRUKER AXS DIFFRAC PLUS software Version 2.0. Samples were prepared for analysis by placing them in a quartz holder.

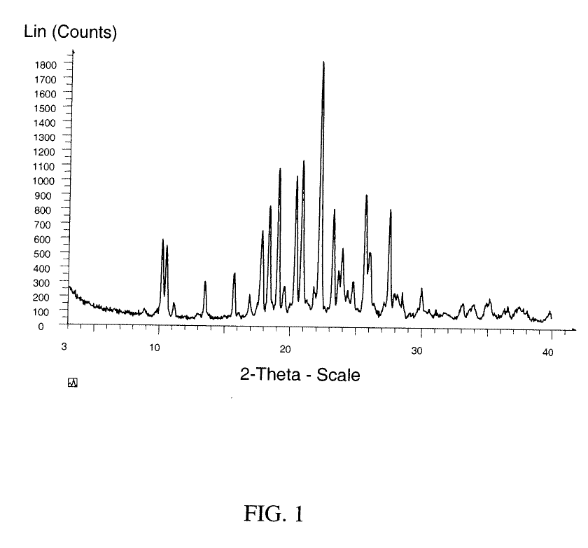

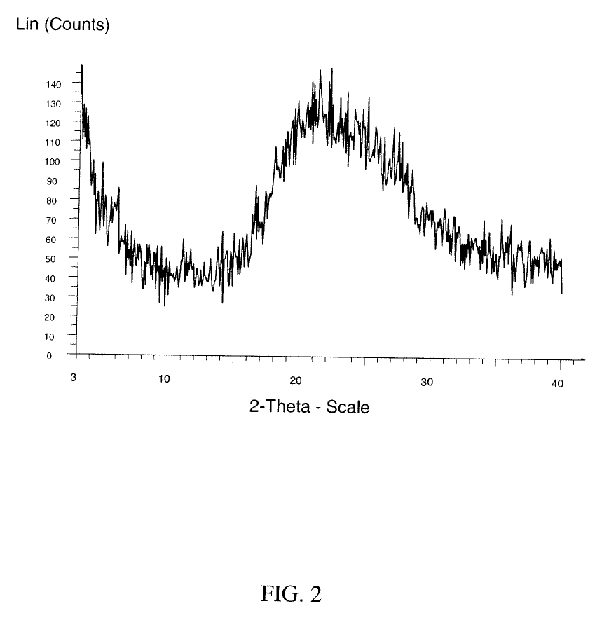

FIG. 1 and FIG. 2 show x-ray powder diffraction (XRPD) patterns for samples of the starting material and Batch A1, respectively. The XRPD diffractogram of the starting material (FIG. 1) is consistent with crystalline Form L of asenapine maleate. The XRPD diffractogram of Batch A1 (FIG. 2) exhibits a single broad peak having a base that extends from about 15° 2θ to about 30° 2θ, which is consistent with an amorphous compound.

Differential Scanning Calorimetry (DSC): Thermograms of samples of the starting material (Form L) and Batch A2 were generated using a METTLER-TOLEDO 822e differential scanning calorimeter under a 60 mL/minute nitrogen purge. Samples were prepared for analysis in 40 μL aluminum pans. The pans were crimped and vented with a pinhole. Data were collected and analyzed using METTLER-TOLEDO STARe software Version 8.10.

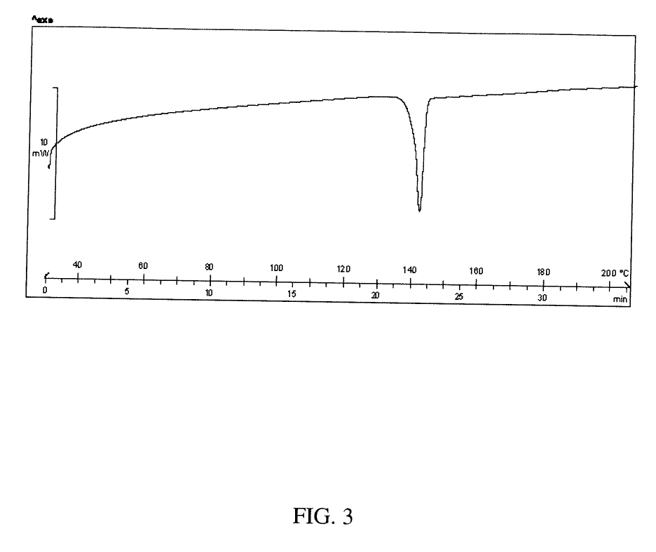

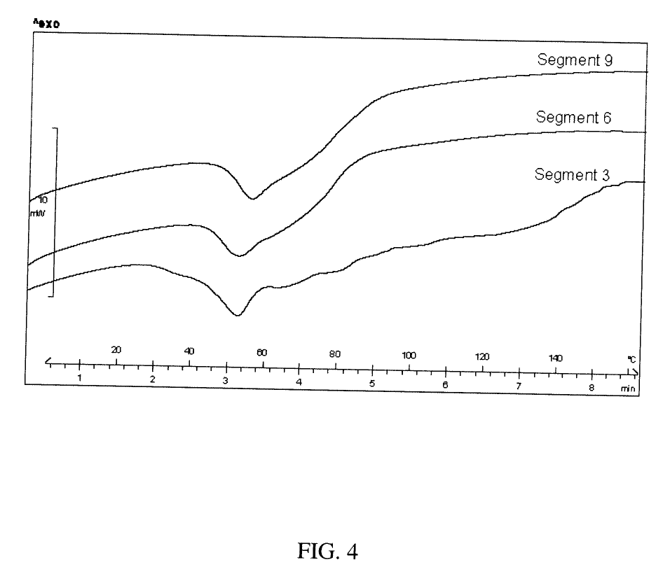

FIG. 3 shows a thermogram for the starting material, which was obtain by heating the sample at a rate of 5° C./minute. As shown in FIG. 3, the starting material exhibits an endothermic event having an onset temperature of approximately 140° C., which is consistent with the melting point of Form L of asenapine maleate. FIG. 4 shows thermograms for Batch A2, during segments 3, 6, and 9 of the following temperature program: (1) cool from 25° C. to −10° C. at 30° C./minute; (2) hold at −10° C. for 5 minutes; (3) heat from −10° C. to 165° C. at 20° C./minute; (4) cool from 25° C. to −10° C. at 30° C./minute; (5) hold at −10° C. for 5 minutes; (6) heat from −10° C. to 165° C. at 20° C./minute; (7) cool from 25° C. to −10° C. at 30° C./minute; (8) hold at −10° C. for 5 minutes; (9) heat from −10° C. to 165° C. at 20° C./minute. FIG. 4 shows that the glass transition temperature (Tg) depends on the thermal history of the sample, since Tg, evaluated during heating segments 3, 6, and 9, is 38.6° C./47.8° C. (onset/midpoint), 43.5° C./48.8° C., and 47.4° C./52.3° C., respectively.

DSC thermograms were also obtained using a different instrument (PerkinElmer Diamond DSC), using amorphous asenapine maleate obtained via rapid cooling of molten drug substance (Form L), and using the following temperature program: (1) heat from 30° C. to 165° C. at 10° C./minute; (2) hold at 165° C. for 5 minutes; (3) quench to −40° C. at 300° C./minute to form amorphous asenapine maleate; and heat from −40° C. to 165° C. at 20° C./minute. Tg evaluated during segment 4 is 52.6° C./54.2° C. (onset/midpoint based on half extrapolated specific heat). From these two analyses, Tg of amorphous asenapine maleate lies in the range of about 38° C. to about 53° C. In addition to the thermal history of the sample, other factors that may influence Tg include the conditions used to generate the amorphous material and the analytical method used to measure Tg.

Solid State Nuclear Magnetic Resonance (ssNMR): An approximately 80 mg sample of the starting material (Form L) or Batch A1 was tightly packed into a 4 mm ZrO₂ spinner. The sample was packed and run under a low humidity environment. The carbon spectrum was collected at ambient temperature and pressure on a BRUKER-BIOSPIN 4 mm BL CPMAS probe positioned into a wide-bore BRUKER-BIOSPIN AVANCE DSX 500 MHz NMR spectrometer. The sample was positioned at the magic angle and spun at 15.0 kHz. The fast spinning speed minimized the intensities of the spinning side bands. The number of scans was adjusted to obtain adequate S/N. The ¹³C solid state spectrum was collected using a proton decoupled cross-polarization magic angle spinning (CPMAS) experiment. The cross-polarization contact time was set to 2.0 ms. A proton decoupling field of approximately 96 kHz was applied; 828 scans were collected. To optimize sensitivity, the recycle delay was adjusted to 3.5 seconds. The carbon spectrum was referenced using an external standard of crystalline adamantane, setting its up field resonance to 29.5 ppm.

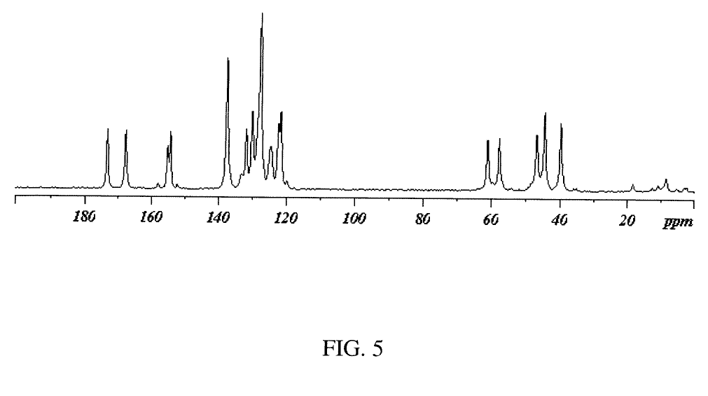

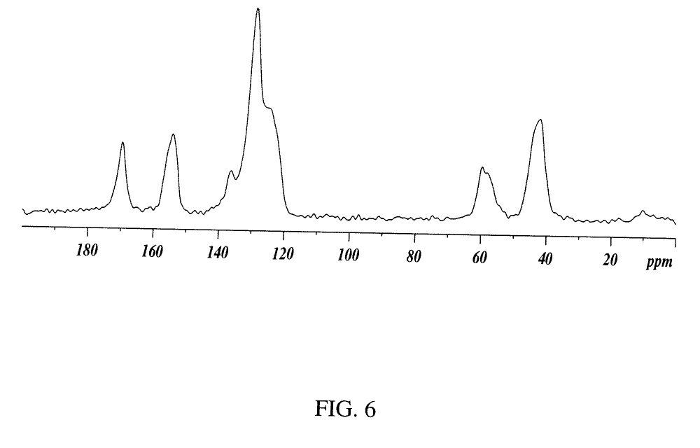

FIG. 5 and FIG. 6 show ¹³C ssNMR spectra collected using CPMAS experiments for samples of the starting material and Batch A1, respectively. TABLE 2, below, lists corresponding carbon chemical shifts (6 in ppm) and peak intensities for the crystalline starting material (Form L) and amorphous asenapine maleate (Batch A1). The chemical shifts are referenced to an external standard of solid phase adamantane at 29.5 ppm, and the intensities are defined as peak heights, which can vary depending on the actual setup of the CPMAS experimental parameters and the thermal history of the sample. The CPMAS intensities are not necessarily quantitative.

TABLE 2
¹³C chemical shifts (δ in ppm) and peak intensities
of crystalline asenapine maleate (Form L) and amorphous
asenapine maleate (Batch A1)

| | |
|---|---|
| Form L | Batch A1 |

| ¹³C δ | | ¹³C δ | |
|---|---|---|---|
| ppm | Intensity | ppm | Intensity |
| 173.4 | 3.9 | 169.9 | 3.86 |
| 168.0 | 3.85 | 154.5 | 4.41 |
| 158.3 | 0.17 | 136.4 | 2.27 |
| 155.4 | 2.78 | 129.5 | 12 |
| 154.6 | 3.77 | 59.9 | 2.78 |
| 152.5 | 0.15 | 58.4 | 2.42 |
| 137.8 | 8.93 | 42.6 | 5.68 |
| 133.3 | 0.89 | | |
| 132.0 | 3.95 | | |
| 130.3 | 5.25 | | |
| 127.9 | 12 | | |
| 124.8 | 2.81 | | |
| 122.5 | 4.37 | | |
| 121.8 | 5.17 | | |
| 120.0 | 0.41 | | |
| 61.1 | 3.33 | | |
| 59.7 | 0.41 | | |
| 57.7 | 3.4 | | |
| 46.9 | 3.73 | | |
| 44.6 | 5.29 | | |
| 39.9 | 4.55 | | |

Relative Humidity (RH): An RH study was conducted to determine appropriate handling conditions for amorphous asenapine maleate. Samples of Batch A1 were placed in double-vial relative humidity chambers at 3.4% RH, 8.2% RH and 22.5% RH and were visually assessed after approximately 36 hours. Samples at 3.4% RH and 8.2% RH remained solid; samples at 22.5% RH deliquesced.

Preparation of Amorphous Asenapine Maleate Via Freeze Drying

Amorphous asenapine maleate is prepared by freeze drying an aqueous solution of asenapine maleate. The solution is prepared by suspending of crystalline asenapine maleate (Form L, 5.50 g) in water (55 mL). To this suspension is added of tert-butanol (55 mL). The resulting solution is filtered through a paper filter, which is rinsed with a small amount of water. The filtrate is freeze dried to provide a fluffy compound (5.48 g).

Tablet Containing Amorphous Asenapine Maleate

A sublingual/buccal dosage form containing amorphous asenapine maleate is formulated with diluents and components to enable rapid tablet disintegration in the mouth. Amorphous asenapine maleate (2 mg) is blended with a sweetener, acesulfame potassium (8 mg), starch (5 mg), and a disintegrant, croscarmellose sodium (5 mg). A small amount of lubricant, magnesium stearate (0.2 mg), is added to improve processing. The blended components are compressed into tablets, which are suitable for delivery via the oral mucosa.

It should be noted that, as used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds.

It is to be understood that the above description is intended to be illustrative and not restrictive. Many embodiments will be apparent to those of skill in the art upon reading the above description, and therefore, the scope of the invention should be determined with to the appended claims, along with the full scope of equivalents to which such claims are entitled. The disclosures of all articles and references, including patents, patent applications and publications, are incorporated herein by reference in their entirety and for all purposes.

1. A compound selected from trans-5-chloro-2-methyl-2,3,3a,12b-tetrahydro-1H-dibenz[2,3:6,7]oxepino[4,5-c]pyrrole and pharmaceutically acceptable complexes, salts, solvates, and hydrates thereof, wherein the compound is at least 50% amorphous based on total weight of the compound.

2. The compound according to claim 1, wherein the compound is at least 75% amorphous based on total weight of the compound.

3. The compound according to claim 1, wherein the compound is at least 90% amorphous based on total weight of the compound.

4. The compound according to claim 1, wherein the compound is at least 95% amorphous based on total weight of the compound.

5. The compound according to claim 1, wherein the compound is at least 99% amorphous based on total weight of the compound.

6. The compound according to claim 1, wherein the compound is a maleic acid salt.

7. The compound according to claim 6, which is characterized by one or more of the following:
a ¹³C solid state nuclear magnetic resonance spectrum, wherein the spectrum includes chemical shifts in parts per million (ppm) of 169.9, 136.4, 129.5, and 42.6, the chemical shifts referenced to an external standard of solid adamantane at 29.5 ppm;
an X-ray powder diffraction pattern obtained with CuKα radiation having a single broad peak between 2θ values of about 15° and about 30°; and
a glass transition onset temperature of about 38° C. to about 53° C.

8. A pharmaceutical composition comprising a compound according to claim 1 or a pharmaceutically acceptable salt thereof, and a pharmaceutically acceptable excipient.

9. A method of making a compound according to claim 1, the method comprising:
forming a liquid solution comprising a solvent and the compound;
atomizing the liquid solution into droplets; and
removing at least a portion of the solvent to form the compound.

10. The method according to claim 9, wherein forming the liquid solution comprises dissolving a precursor of the compound in the solvent, the precursor having the same chemical structure as the compound but less amorphous content.

11. The method according to claim 10, wherein the precursor is at least 99% crystalline based on the total weight of the precursor.

12. The method according to claim 10, wherein the precursor is a maleic acid salt of trans-5-chloro-2-methyl-2,3,3a,12b-tetrahydro-1H-dibenz[2,3:6,7]oxepino[4,5-c]pyrrole.

13. A method of treating a condition or disorder in a subject, the method comprising administering to the subject in need of treatment a therapeutically effective amount of a compound according to claim 1 or a pharmaceutically acceptable salt thereof, wherein the disorder or condition is selected from schizophrenia and other psychotic disorders, mood disorders, and combinations thereof.

14. The method of claim 13, wherein the disorder is schizophrenia.
